(19) United States
(12) Patent Application Publication
(10) Pub. No.: US 2005/0283174 A1
(43) Pub. Date: Dec. 22, 2005

(54) Vein stripping tool and method of use

(76) Inventors: John C. Opie, Scottsdale, AZ (US); Stephen J. Joyce, Phoenix, AZ (US)

Correspondence Address: SQUIRE, SANDERS & DEMPSEY L.L.P.;Suite 2700, Two Renaissance Square, 40 North Central Avenue, Phoenix, AZ, 85004-4498, US

(21) Appl. No.: 11/046,218

(22) Filed: Jan. 27, 2005

Related U.S. Application Data
(60) Provisional application No. 60/539,404, filed on Jan. 27, 2004.

(51) Int. Cl. A61B 17/22

(52) U.S. Cl. 606/159

(57) ABSTRACT

A vein stripping device and method of use are disclosed. One embodiment of the vein stripping device comprises an endovascular component having a first endovascular end and a second endovascular end, a handle that attaches to one of the endovascular ends and a vein stripper that attaches to the endovascular end opposite the handle. The endovascular component is sized to fit inside the lumen of a to-be-removed vein. The vein stripper preferably includes an internal cavity for collecting at least a portion of the to-be-removed vein and preventing at least a portion of the to-be-removed vein from slipping over the vein stripper. In use, a vein is accessed in two locations and the endovascular component is positioned in the vein lumen. The handle is attached to one endovascular end and the vein stripper to the second endovascular end. A user then pulls the handle and pulls the vein stripper along the length of the accessed vein thus removing the vein.

### RELATED APPLICATIONS

This application claims priority to U.S. Provisional Application Ser. No. 60/539,404, filed Jan. 27, 2004, by inventors John C. Opie and Stephen J. Joyce, and entitled “Vein Stripping Tool and Method of Use,” which is incorporated herein by reference, and to U.S. Utility application Ser. No. 10/868,746 filed Jun. 14, 2004 and entitled “Method of Stripping Diseased Blood Vessels from the Human Body,” the disclosure of which is incorporated herein by reference.

### TECHNICAL FIELD OF THE INVENTION

This invention relates to the field of surgical instruments and more specifically to a vein stripping device and method for stripping a diseased vessel from a body.

### BACKGROUND OF THE INVENTION

Veins are tubular blood vessels that carry blood back to the heart. Veins that are located below the heart, especially those in the leg, have to return the blood to the heart against gravitational forces (similar to pumping water uphill). Veins have internal structures, known as valves, which keep the flow of blood going in the right direction towards the heart. The valves operate as check valves, opening to allow flow towards the heart and closing to prevent blood from being pulled by gravity towards the foot. However, if these valves become weak and allow at least partial reverse flow, the valves of the vein are said to have valvular incompetence, also known as varicose veins. Varicose veins are common; it is estimated that about 80 million people in the United States at any one time suffer from some form of varicose venous disease.

Significantly diseased varicose veins cause considerable problems. First, varicose veins can be unsightly. Varicose veins tend to bulge visibly when the person is standing. Also, older varicose veins can cause a steady buildup of iron in the subcutaneous fat layers and skin of the lower leg, resulting in permanent staining of the tissues. This is known as hemosiderosis and can result in substantial local pain and unsightly rust colored skin depressions from fat necrosis.

Not only are varicose veins a problem aesthetically, varicose veins can cause a number of other health problems. Varicose veins can be painful when the person is standing. Additionally, irritation to a varicose vein can result in a blood clot near the surface of the skin. This is known as superficial thrombophlebitis and can be a very painful condition. It is also possible that a varicose vein sufferer may get a blood clot deeper in the vein, a condition known as deep vein thrombophlebitis (DVT). Clots formed from DVT may circulate through out the body and form a blockage in an artery of the lung. This condition is known as pulmonary embolus and can result in sudden death.

With very superficial, high-pressure, large, varicose veins it is possible that a minor skin abrasion in an area near the shin can cause the vein to rupture and, in rare cases, threaten a patient's life from loss of blood, or exsanguinations. Also, the backflow of blood to the foot area can result in deoxygenated blood leaking out from the blood vessels in the feet, causing edema. The tissue in that area can no longer receive oxygenated blood and venous stasis ulcers can form. Advanced venous stasis ulcers can be a very challenging problem to resolve.

Because of the problems that varicose veins present, it is in the best interests of the patient to prevent the progression from early varicose veins to advanced complicated varicose veins and the formation of venous stasis ulcers. Once it is established that the patient has sapheno-femoral valvular incompetence (varicose vein) and consequently a backflow of blood through the damaged valve of a vein, such as the greater saphenous vein, then a greater saphenous vein stripping is a recommended procedure that can prevent most of the advanced complications from developing over time.

Vein stripping dates back to at least the beginning of the twentieth century and the work of William Wayne Babcock. In a typical stripping of the greater saphenous vein (GSV), the greater saphenous vein is exposed using an incision in the groin area, and delivering the greater saphenous vein at its junction with the common femoral vein, just below the inguinal ligament. Once delivered, the GSV is ligated in continuity. The GSV is then exposed in front of the medial malleolus at the ankle. Once delivered, the GSV at the ankle is also ligated in continuity and divided. A flexible plastic stripper cable is then passed up the GSV and is variably threaded to the groin section of the GSV. On occasions it is not possible to pass the stripper cable due to knots and snarls within the GSV. Secondary incisions then become necessary and the process repeats. Once at the groin, a ligature is passed around the GSV and loosely tied to prevent excessive blood loss. At this time the GSV is now divided and the terminal expansion of the stripper cable is passed out of the vein. At this time the second ligature is firmly tied around the stripper cable. A stripper head is now attached to the stripper cable.

In the method of Babcock/Meyer the stripper head is similar in shape to half an acorn with a flat leading surface and comes in three sizes, large, medium and small. Typically, the medium size is selected. The stripper head is fitted into the groin incision and the vein is then avulsed from the leg from a longitudinal pull from the ankle incision using some form of a handle attached to the stripper cable. The vein bunches up against the leading surface of the stripper head as the stripper head travels from the groin area to the ankle area. In order to reduce bleeding as the vein is stripped, the leg may be either compressed with a firm ACE bandage around the leg before the strip is performed or local pressure can be applied as the stripper head passes down the leg. Once that is done the two or more incisions are suture closed and the patient is sent home after several recovery hours with an ACE bandage around the leg. Three or four days later the patient usually visits the doctor's offices and the ACE wrap is removed. More commonly than not, the leg has significant bleeding below the skin or ecchymoses. Cords of contained hematomas may exist down the line where the veins were stripped and the patient typically has a painful, swollen leg. The bruising takes several weeks to resolve as does the discoloration from the post-operative hemorrhage that usually occurs. Sometimes it is necessary to return the patient to the operating room to surgically release pressurized, painful, hematomas.

The traumatic component of the Babcock/Meyers technique can be easily understood by understanding how the stripper head is designed. It is similar to a toadstool shape with a cone like trailing end but a flat or blunt leading end that must be pulled through the patient's tissues. Pulling such a blunt ended instrument through the patient's leg from the groin to the ankle is a traumatic event, as considerable force is required to pull the flat cone down the full length of the leg. As larger stripper heads are selected, the pulling force required rises substantially. The reverse is obviously also true. The smaller head, however, runs a risk that the vein will flow over the stripper head and not be stripped.

Other prior art solutions to varicose veins involve non-stripping procedures. Such procedures include radio frequency ablation, laser ablation and sclerotherapy. The former two procedures produce clots in the varicose vein by heat. In the case of sclerotherapy clot formation may occur from the use of blood coagulation chemicals. Each procedure has the potential to produce pulmonary embolism, which can lead to sudden death. The main problem with these techniques is centered around the continued existence of the varicose vein. That is, the vein remains in situ and can remain unsightly. In all of these techniques there is deep pain, risk of pulmonary embolism and recurrence or rechannelization of the veins. A significant number of patients will then revert to a traditional stripping technique of Babcock and Meyers.

Prior art techniques also include inversion stripping, also known as invagination stripping. In this situation, the endovenous cable is passed as before up the vein but no stripper head is attached. Rather, the vein is simply tied to the cable before the expansion. The vein is now pulled out of the leg and inverts on itself as it does so. The main problem with this technique is that as the vein inverts, the bulky, large tubular part of the vein, which lies in the groin and thigh must fit inside the smaller, less bulky part of the vein in the calf area. Sometimes, it is impossible for the inversion technique to continue and the vein is simply torn apart and the stripping stops. That immediately results in bleeding and necessitates an additional incision to locate the unstripped vein portion, which must be retrieved and stripped. Thus, while invagination stripping is less traumatic that the Babcock/Meyers method, it is less effective in facilitating successful stripping of varicose veins. For this reason the Babcock/Meyers stripping procedure has become the procedure of common use. The essential problem with the Babcock/Meyers procedures is (1) trauma and (2) postoperative bleeding and its complications.

### SUMMARY OF THE INVENTION

The present invention addresses this problem by permitting a smaller head without running any risk of the vein flowing over the head. Such a concept substantially diminishes the trauma. The post-operative hemorrhage tendency is ever existent. The present invention also addresses this problem.

In one embodiment a vein stripping device comprises an endovascular component having a first end and a second end, a handle that attaches to one end of the endovascular component and a vein stripper that attaches to the other end of the endovascular component. The endovascular component is sized to fit inside the lumen of a to-be-removed vein. The vein stripper includes an internal cavity for collecting at least a portion of the to-be-removed vein and preventing the to-be-removed vein from slipping over the vein stripper.

In another embodiment the first end and the second end are generally ovoid in shape. The ovoid shape facilitates the movement of the endovascular component through the lumen of the to-be-removed vein. Additionally, the vein stripper can have a sloping leading edge adjacent to the internal cavity and a sloping trailing edge to facilitate the movement of the vein stripper through the body.

In another embodiment the vein stripper includes a projection operable to receive and retain a wound drain. As the vein stripper is pulled and the vein stripped, the trailing wound drain is left in the body in order to drain bodily fluids and help prevent the formation of clots or hematomaes.

In another embodiment a method for stripping a diseased vein from the body is disclosed. In one exemplary method, a first end of the diseased vein is first opened near a groin incision. Next, a second end of the diseased vein is opened near an ankle incision. An endovascular component having a first endovascular end and a second endovascular end is fed through the lumen of the diseased vein from the second end of the diseased vein to the first end of the diseased vein. A handle is then attached to one end of the endovascular component and a vein stripper is attached to the second end of the endovascular component. The vein stripper includes an internal cavity at a first end. A wound drain is attached to a projection located at a second end of the vein stripper. The endovascular component is then pulled using the handle, forcing the vein stripper down the diseased vein. At least part of the diseased vein collects in the internal cavity. The vein stripper travels down the vein to the first end of the vein at the ankle incision. The vein stripper is removed, the wound drain is exteriorized by attaching a conduit thereto and all incisions are closed.

### BRIEF DESCRIPTION OF THE PREFERRED EMBODIMENTS

These and other aspects, features and advantages of the present invention will become apparent from the following description of the invention in reference to the appended drawing in which like numerals denote like elements and in which:

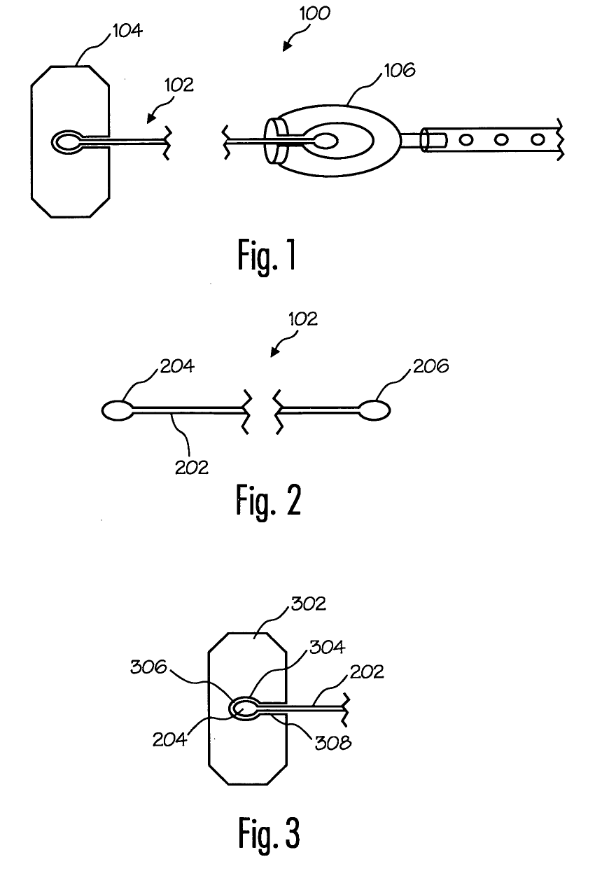

FIG. 1 is a view of a vein stripping device according to the invention;

FIG. 2 is a view of the endovascular component of the vein stripping device;

FIG. 3 is a view of the handle of the vein stripping device with one end of the cable installed;

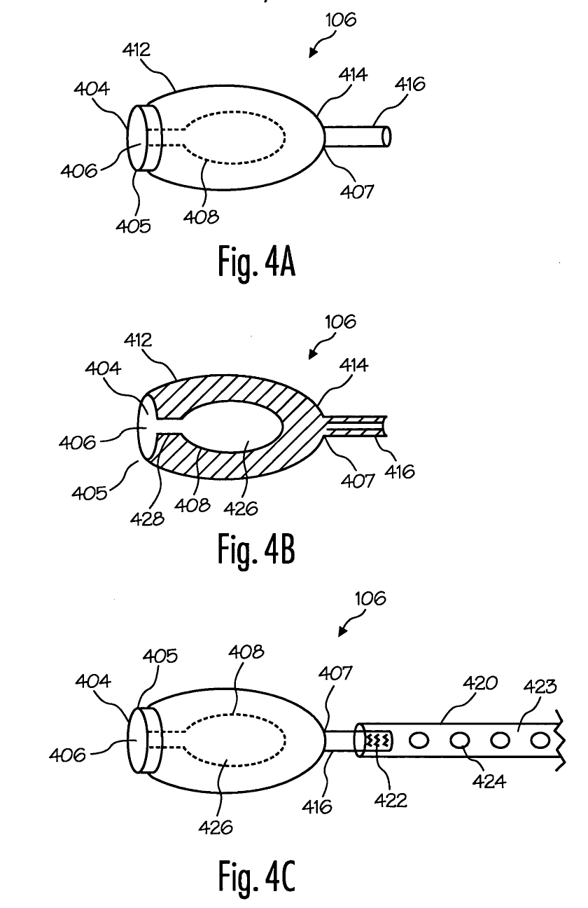

FIG. 4a is a view of the vein stripper, FIG. 4b is a cut away view of the vein stripper and FIG. 4c is a view of the vein stripper with an end of the endovascular component installed;

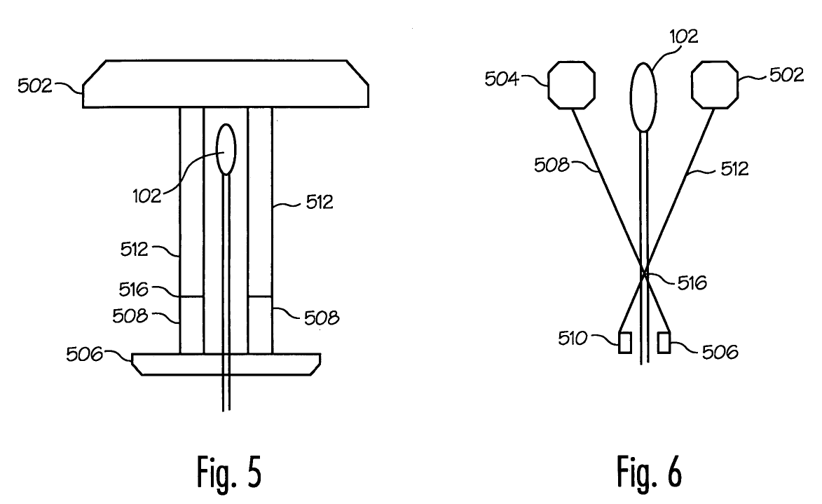

FIG. 5 is a view of an alternate handle for the vein stripper;

FIG. 6 is a side view of FIG. 5;

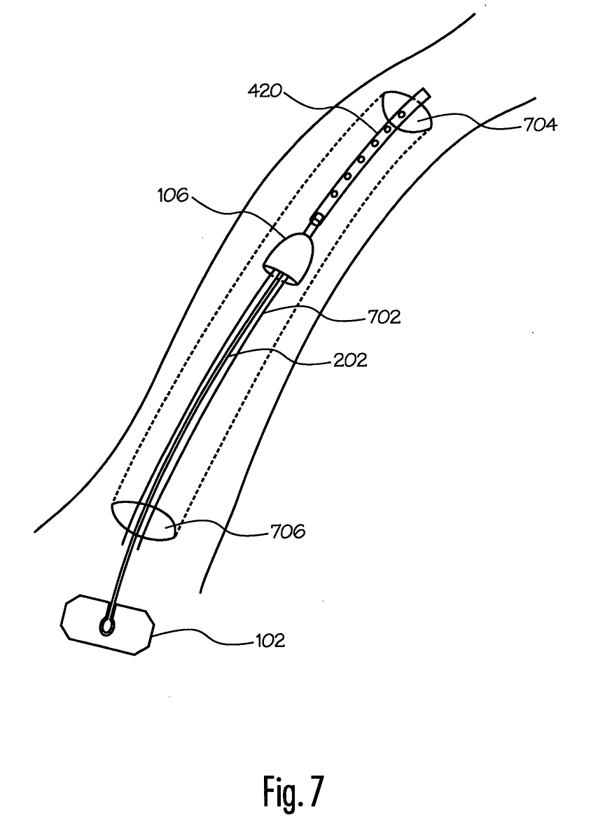

FIG. 7 illustrates a vein with the present invention being used to strip the vein; and

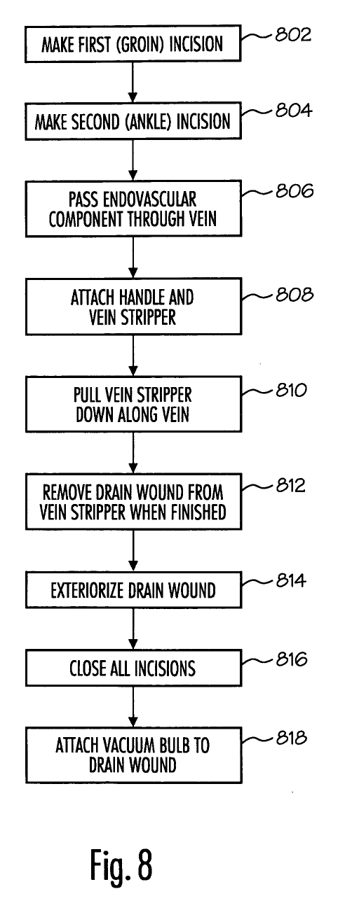

FIG. 8 is a flowchart for a method to remove a varicose vein from the body.

### DETAILED DESCRIPTION OF THE PREFERRED EMBODIMENTS

In the following descriptions the vein stripping device is discussed in the context of removal of varicose veins. However, the device and method of the present invention can be used to remove other diseased veins and tubular body parts. In the context of this invention, a vein stripping device “strips” or removes a diseased vein from the body, typically in such a manner that destroys the vein. This is in contrast to a vein harvesting device which removes a vein from the body in such a manner that it can be reused in another location.

A varicose vein remover, in accordance with an embodiment of the invention, preferably includes three parts: (1) an endovascular component, which may be a flexible or semi-rigid plastic cable, that is positioned inside the vein lumen, preferably by being passed through the lumen, the endovascular device preferably having ends configured to engage a handle and/or a vein stripper; (2) a handle that couples to one of the ends of the endovascular device; and (3) a vein stripper that couples to the end of the endovascular device opposite the handle and which can be used to remove the vein.

FIGS. 1-4 illustrate components of an exemplary vein stripping device 100. Vein stripping device 100 is comprised of an endovascular component 102 which is fed through the interior of the to-be-removed vein, a vein stripper 106, which is attachable to one end of endovascular component 102 and that gathers up at least part of the to-be-removed vein as vein stripper 106 is pulled down the to-be-removed vein, and a handle 104, which is attachable to an opposite end of the endovascular component 102. Handle 104 is used to exert a force on endovascular component 102 in order to pull vein stripper 106 (when attached to endovascular component 102) down the to-be-removed vein.

Endovascular component 102 is any device capable of being passed through the inside of the vein or other tubular body part to be removed. In one embodiment, as seen in FIG. 2, endovascular component 102 is comprised of a long section of a flexible, or semi-rigid, cable 202 having a first expansion 204 at one end and a second expansion 206 at the other end. First expansion 204 and second expansion 206 are shaped such that they can be inserted into a lumen of a vein and passed through the lumen of the vein without damaging the inner layers of the vein. In one embodiment first expansion 204 and second expansion 206 are generally ovoid, or egg-like, in shape. The curved surface of first expansion 204 and second expansion 206 aid in the insertion and movement of endovascular component 102 through the lumen of the vein. In one embodiment, first expansion 204 and second expansion 206 are identical in size and shape so that either end can be used as the leading edge to pass up a vein.

In one embodiment, cable 202, the first expansion 204 and the second expansion 206 are made of any bio-compatible plastic. Endovascular component 102 is long enough to be passed through a vein with sufficient length left over to extend out of the vein so that vein stripper 106 can be coupled to one end of the endovascular component 102 and the handle 104 to another end of the endovascular component 102. In one embodiment, endovascular component 102 is approximately 100 cm long, about a third longer than the length of an average leg, which is approximately 70 cm. The first and second expansions 204 and 206 are approximately 3 mm wide at the widest point. Cable 202 is approximately less than 3 mm wide.

Handle 104 couples to endovascular component 102 and provides a user (typically a surgeon) with a structure to grasp in order to exert a pulling force on the endovascular component 102. Handle 104, shown in FIG. 3, includes a grip portion 302 and an installation slot 304. Grip portion 302 provides a contact surface for a user to grasp when exerting a force to pull vein stripper 106 down the vein. Grip portion 302 can be made from any suitable material. Grip portion 302 can be of any size or shape that allows a user of vein stripper 106 to grip the handle 104 and apply sufficient force to strip the vein. Grip portion 302 can be sized to accommodate one-or two-handed operation. Grip portion 302 can optionally be coated with, covered by, or made from a material that enhances the friction between the surgeon's hands and the handle.

Installation slot 304 provides an area where an endovascular end of endovascular component 102 can be inserted to attach handle 104 to endovascular component 102 in order to allow a surgeon to pull on handle 104 to exert a force. Any suitable structure may be used for this purpose, however.

In one embodiment, installation slot 304 comprises a slot in handle 104 that extends from the surface of handle 104 towards and approximately into the middle of handle 104. Installation slot 304 has (a) an expansion-shaped section 306 for receiving one of either first expansion 204 or second expansion 206 of endovascular component 102, and (b) a cable- or rod-shaped section 308 for accepting a portion of cable 202 of endovascular component 102 near the expansion. To attach handle 104 to endovascular component 102, either first expansion 204 or second expansion 206 of endovascular component 102 is inserted into the expansion-shaped section 306 of installation slot 304 and cable 202 of endovascular component 102 adjacent to the expansion is inserted into cable-shaped section 308 of installation slot 304. First extension 204 and second extension 206, and part of cable 202 of endovascular component 102 are now inserted into handle 104. Force exerted on handle 104 along essentially the same vector as defined by endovascular component 102 results in a pulling force on endovascular component 102, and in this embodiment assists in securing handle 104 to the expansion (204 or 206) of cable 202 to which handle 104 is attached.

While installation slot 304 provides one way of connecting endovascular component 102 to handle 104, any other method of coupling handle 104 to endovascular guide 102 can be used without departing from the scope of the present invention. For example, endovascular component 102 may screw-on, snap-on, bolt on or otherwise be attached to handle 104.

Vein stripper 106 attaches to the end of endovascular component 102 opposite the end to which handle 104 attaches. Vein stripper 106, when attached to endovascular component 102, can be pulled along the vein to be removed, causing at least part of the vein to crumple in the internal cavity of vein stripper 106. Vein stripper 106, as seen in FIGS. 4a-4c, includes an opening 404 at one end 405, the opening 404 leading to an internal cavity 406. Vein stripper 106 also includes a stripper installation slot 408 for receiving an end of endovascular component 102 in order to attach endovascular component 102 to vein stripper 106.

Vein stripper installation slot 408, in one embodiment, is similar to installation slot 304 in that it is a slot where an end of the endovascular component can be inserted to couple vein stripper 106 to endovascular component 102, and any suitable structure may be used for this purpose. As shown, vein stripper installation slot 408 comprises a vein stripper expansion-shaped section 426 that receives one of either the first expansion 204 or second expansion 206 of endovascular component 102 and a vein stripper cable- or rod-shaped section 428 that receives a portion of cable 202 of endovascular component 102. In this embodiment, one end of endovascular component 102 is inserted into vein stripper insertion slot 408. Vein stripper installation slot 408 extends from the surface of vein stripper 106 to inside vein stripper 106. In one embodiment, vein stripper installation slot 408 extends from the surface of vein stripper 106 to approximately the middle of vein stripper 106. This allows endovascular component 102 to be firmly coupled to handle 104, especially when force is applied to handle 104.

As discussed previously, the attachment system discussed above is only one example of how endovascular component 102 can be connected to vein stripper 106. Other methods of connecting endovascular component 102 to vein stripper 106 can be used without departing from the scope of the present invention. For example, endovascular component 102 may screw-on, snap-on, bolt on or otherwise attach to vein stripper 106.

Vein stripper 106 is configured to travel through the inside of the body when pulled by a cable or similar device in such a way as to reduce trauma in the body. To reduce trauma, in one embodiment vein stripper 106 is generally ovoid in shape, with a sloping leading edge 412 and a sloping trailing edge 414 of the surface. In addition to reducing trauma, the sloping leading and trailing edges 412 and 414 reduce the force required to pull vein stripper 106 through the body as compared to prior vein strippers, such as the acorn shaped stripper head used in the Babcock Procedure.

Internal cavity 406 of the vein stripper 106 catches at least part of the to-be-removed vein as vein stripper 106 is pulled down the vein. In one embodiment, vein stripper installation slot 408 is extended to approximately the middle of vein stripper 106 so the to-be-removed vein will be coaxial with opening 404 of vein stripper 106. Since at least part of the vein collects in internal cavity 406 the vein can not pass over vein stripper 106 as it can in other vein removal procedures, such as the Babcock technique, wherein a vein can sometimes pass over the stripper head, stopping the procedure. This is especially a problem when smaller Babcock-type strippers (acorn shaped) are used. By preventing the to-be-removed vein from passing over vein stripper 106, vein stripper 106 can be smaller in size as compared to other prior art stripper heads. A smaller stripper head helps to reduce the trauma to the body when stripping the vein.

Vein stripper 106 may also include an optional projection 416 at a second end 407. A wound drain 420 may be connected to projection 416. Projection 416 can optionally include securing devices 422 to assist in securing wound drain 420 to projection 416. Securing device 422 can be any structure capable of assisting in securing wound drain 420 to projection 416, such as a series of chevrons that increases the frictional force between projection 416 and wound drain 420.

Wound drain 420 can be any device designed to promote the removal of blood, clots and other bodily fluids from an area of the body. Wound drain 420 is typically a cylindrical or squared shaped structure with a central lumen (not pictured) and a number of openings 424 on the surface 423 of the wound drain 420 that extend to the central lumen. The wound drain 420 is typically made from a flexible material such as silicon, and some drains that may be used with the invention are disclosed in co-pending U.S. application Ser. No. 10/863,009 to John C. Opie, Stephen J. Joyce, and Thomas Izdebski, entitled “Surgical Drains,” filed on Jun 7, 2004, the disclosure of which is incorporated herein by reference.

Attaching wound drain 420 to projection 416 at second end 407 of vein stripper 106 allows for wound drain 420 to be pulled through the body along with vein stripper 106. In this manner wound drain 420 is automatically placed in the subcutaneous area formed when the vein is removed. When vein stripper 106 is removed from the body, wound drain 420 is separated from vein stripper 106. Wound drain 420 is then in place to help remove blood and other bodily materials from the area where the vein was removed.

Handle 104 in the previous embodiment of the invention attaches to one end of the endovascular component 102. In the removal of a long vein such as the greater saphenous vein (GSV), vein stripper 106 will move from approximately the groin area to approximately the ankle area. Handle 104 attaches to the end of the endovascular component 102 near the ankle and vein stripper 106 starts near the groin. As the surgeon pulls on handle 104, vein stripper 106 moves down the vein and collects at least part of the vein in the internal cavity 406. But, since handle 104 is fixed to the end of the endovascular component 102, the user has to move farther and farther away from the ankle incision area as the handle is pulled. This can be inconvenient for the user and makes vein removal more difficult.

To alleviate this problem, an alternative handle can be used. Referring to FIGS. 5-6, movable handle 500 includes a top handle 502, a bottom handle 504, a top gripper pad 506 connected to bottom handle 504 by one or more first arm(s) 508 and a bottom gripper pad 510 connected to top handle 502 by one or more second arm(s) 512. First arms 508 and second arms 512 are connected at a connection point 516.

Top handle 502 and bottom handle 504 provide a surface area for the surgeon to grasp when exerting a pulling force to move vein stripper 106. Top handle 502 and bottom handle 504 can be made of any suitable material that provides a convenient surface for a surgeon or other user to move moveable handle 500 during surgery. For example, top and bottom handle 502 and 504 can be made of any hard material such as a rigid plastic material or the like.

Top gripper pad 506 and bottom gripper pad 510 holds an object, such as the cable 202 of endovascular component 102, to maintain the grip. Top gripper pad 506 and bottom gripper pad 510 are preferably made from any material that can secure to an object without slipping.

First arm 508 and second arm 512 can be any structure that can couple bottom handle 504 and top handle 502 to top and bottom gripper pads 506 and 510, respectively. In one embodiment, first arm 508 and second arm 512 are made of metal and plastic, although any suitable material may be used. In one embodiment, there are two first arms 508 and two bottom arms 512 that couple the top and bottom handles 502 and 504 to the top and bottom gripper pads 506 and 510. First arms 508 connect to second arms 512 at connection point 516. The location of the connection point 516 is chosen to provide a multiplying effect to the force exerted on top and bottom handles 502 and 504. This reduces the amount of force required to release the top and bottom gripper pads 506 and 510 from cable 202.

The connection point 516 allows for the up and down movement of first arm 508 and second arm 512 while coupling first arm 508 and second arm 512. In one embodiment, connection point 516 includes a spring tension such that the top gripper pad 506 and bottom gripper pad 510 contact cable 202 of endovascular component 102 when the moveable handle 500 is in a relaxed state.

When in a relaxed state, that is, when no pressure is applied to the handle, top gripper pad 506 and bottom gripper pad 510 contact endovascular component 102, securing moveable handle 500 to the cable 202 of the endovascular component 102. To move the moveable handle 500, the surgeon compresses top handle 502 and bottom handle 504 towards each other, causing the top gripper pad 506 and the bottom gripper pad 510 to move apart, releasing their hold on the cable 202. In this manner, the surgeon can keep the moveable handle 500 closer to the incision.

The present invention can be used to remove damaged veins such as a varicose vein. FIG. 7 illustrates the vein stripping device 100 in use. In FIG. 7, vein stripping device 100 is shown removing a greater saphenous vein 702. In this embodiment, a first incision 704 is made near the groin to expose one end of the vein 702. A second incision 706 is made near the ankle to expose a second end of the vein. As seen in FIG. 7, the vein stripper 106 travels inside the body, crumpling the vein as it travels. Wound drain 420 (if used) trails behind vein stripper 106.

An exemplary method for using vein stripping device 100 to remove a varicose vein is shown as a flow chart in FIG. 8. In a first step 802, first incision 704 is made near the groin of the patient and the GSV is located and divided. In step 804, the second incision 706 is made near the ankle to expose the greater saphenous vein 702 at that location.

Next, in step 806, the endovascular component 102 is passed through vein 702 to be removed. The endovascular component 102 is sufficiently flexible to allow passage through vein 702. Typically, endovascular component 102 is fed from the second (ankle) incision 706 to the first (groin) incision 704 until part of endovascular component 102 exits the first (groin) incision 704.

In step 808, vein stripper 106 and handle 104 are attached to first expansion 204 and second expansion 206 of endovascular component 102. Vein stripper 106 is attached at the endovascular end of endovascular component 102 near the first (groin) incision 704 and the handle 104 is attached to the endovascular end of endovascular component 102 near the second (ankle) incision 706. Vein stripper 106 is aligned such that the opening of vein stripper 106 will contact vein 702 as it is pulled by endovascular component 102, which moves when force is applied to handle 104.

The user then pulls on handle 104, in step 810, moving vein stripper 106 down the to-be-removed vein 702. Vein 702 crumples and collects at least partially in the internal cavity 406 of the vein stripper 106. Also, as vein stripper 106 moves down the vein, the wound drain 420 trails behind.

When vein stripper 106 reaches the second (ankle) incision 706, in step 812, vein stripper 106 is separated from the wound drain 420. In step 814, wound drain 420 is exteriorized. The first (groin) and second (ankle) incisions 704-706 are closed in step 816. Then, in step 818, wound drain 420 is attached to a vacuum bulb (not pictured) to remove blood, clots or other bodily debris.

Any vein could theoretically be removed using this method and device(s). Further, all that is necessary to perform a method according to the invention is to make one or more incisions to access a length of vein and to pull the vein stripper along the vein to strip the vein. It is preferred that the endovascular component be inserted into the vein to (relatively) straighten it and guide the vein stripper.

Having now described preferred embodiments of the invention modifications and variations may occur to those skilled in the art. The invention is thus not limited to the preferred embodiments, but is instead set forth in the following claims and legal equivalents thereof. Unless expressly stated in the claims or specification, method steps may be performed in any order capable of yielding the end result.

1. A method for stripping a vein from a body comprising:
accessing a first end of the vein;
accessing a second end of the vein;
positioning an endovascular component having a first endovascular end and a second endovascular end into a lumen of the vein;
attaching a handle to the first endovascular end near the first end of the vein;
attaching a vein stripper to the second endovascular end near the second end of the vein, the vein stripper having an internal cavity;
pulling the endovascular component using the handle, and thereby pulling the vein stripper from the first end of the vein to the second end of the vein, wherein at least part of the vein collects in the internal cavity.

2. The method of claim 1 wherein the step of attaching the handle further comprises inserting one of the endovascular ends into a handle installation slot.

3. The method of claim 1 wherein the step of attaching the vein stripper further comprises inserting one of the ends of the endovascular component into a vein stripper installation slot.

4. The method of claim 1 wherein the vein stripper has an internal cavity at one end for collecting at least a portion of the to-be-removed vein and preventing at least some of the to-be-removed vein from slipping over the vein stripper.

5. The method of claim 1 wherein the vein stripper has a sloping leading edge that reduces trauma to the body as the vein stripper strips the vein.

6. The method of claim 1 wherein the vein stripper has a sloping trailing edge to reduce trauma to the body.

7. The method of claim 1 wherein the vein stripper has a projection formed at a second end, the projection configured to receive a wound drain.

8. The method of claim 1 wherein the vein is the GSV.

9. The method of claim 8 wherein the first incision is made in the groin area.

10. The method of claim 8 wherein the second incision is made in the ankle area.

11. The method of claim 1 that further includes attaching a wound drain to the second endovascular end.

12. The method of claim 11 that further comprises the step of detaching the wound drain from the second endovascular end and leaving it in a location that had been occupied at least partially by the vein.

13. The method of claim 12 further comprising the step of attaching a vacuum to the wound drain to help remove bodily fluid.

14. The method of claim 12 that further includes the step of connecting a first end of a conduit to the drain.

15. The method of claim 14 that further includes the step of connecting a second end of the conduit to a vacuum.

16. The method of claim 1 that further includes the step of closing all incisions.

17. The method of claim 11 wherein the step of attaching a wound drain further comprises attaching a wound drain to a projection of the vein stripper, the projection having one or more securing devices to assist in the attachment and retention of the wound drain.
